(19) United States
(12) Patent Application Publication
(10) Pub. No.: US 2004/0235147 A1
(43) Pub. Date: Nov. 25, 2004

(54) System, method, and encased probe array product

(75) Inventor: John C. Chappell, Morgan Hill, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/851,050

(22) Filed: May 21, 2004

Related U.S. Application Data
(60) Provisional application No. 60/472,798, filed on May 21, 2003.

(51) Int. Cl. C12M 1/34

(52) U.S. Cl. 435/287.2; 435/288.7

(57) ABSTRACT

In one embodiment an encased probe array is described. The encased probe array comprises a first layer including one more active areas disposed thereon, where each of the one or more active areas includes a plurality of probes each enabled to hybridize a biological molecule; one or more second layers attached to the first layer; and one or more chambers formed from at least one of the one or more second layers, where each of the one or more active areas is associated with at least one of the one or more chambers.

### RELATED APPLICATIONS

The present application claims priority to U.S. Provisional Patent Application Serial No. 60/472,798, titled “Integrated Glass Cartridge”, filed May 21, 2003, which is hereby incorporated by reference herein in its entirety for all purposes.

### BACKGROUND

1. Field of the Invention

The present invention relates to the examination of biological materials, more particularly providing an encased probe array that includes one or more active areas, each comprising probes enabled to hybridize a biological molecule.

2. Related Art

Synthesized nucleic acid probe arrays, such as Affymetrix® GeneChip® probe arrays, and spotted probe arrays, have been used to generate unprecedented amounts of information about biological systems. For example, the GeneChip® Human Genome U133 Plus 2.0 probe array available from Affymetrix, Inc. of Santa Clara, Calif., is comprised of a single microarray containing over 1,000,000 unique oligonucleotide features covering more than 47,000 transcripts that represent more than 33,000 human genes. Analysis of expression data from such microarrays may lead to the development of new drugs and new diagnostic tools.

### SUMMARY OF THE INVENTION

Systems, methods, and products to address these and other needs are described herein with respect to illustrative, non-limiting, implementations. Various alternatives, modifications and equivalents are possible. For example, certain systems, methods, and computer software products are described herein using exemplary implementations for analyzing data from arrays of biological materials produced by the Affymetrix® 417™ or 427™ Arrayer. Other illustrative implementations are referred to in relation to data from Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

In one embodiment an encased probe array is described. The encased probe array comprises a first layer including one more active areas disposed thereon, where each of the one or more active areas includes a plurality of probes each enabled to hybridize a biological molecule; one or more second layers attached to the first layer; and one or more chambers formed from at least one of the one or more second layers, where each of the one or more active areas is associated with at least one of the one or more chambers.

Also, a method of producing an encased probe array is described. The method comprises disposing one more active areas on a first layer, where each of the one or more active areas includes a plurality of probes each enabled to hybridize a biological molecule; forming one or more chambers from at least one of one or more second layers, wherein each of the one or more active areas is associated with at least one of the one or more chambers; and attaching one or more second layers to the first layer.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures or method steps and the leftmost digit of a reference numeral indicates the number of the figure in which the referenced element first appears (for example, the element 100 appears first in FIG. 1). In functional block diagrams, rectangles generally indicate functional elements and parallelograms generally indicate data. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a computer system, a scanner, a hybridization station, and an encased probe array;

FIG. 2 is a functional block diagram of one embodiment of the computer and encased probe array of FIG. 1;

FIG. 3A is a simplified graphical representation of one embodiment of the encased probe array of FIGS. 1 and 2 including active areas positionally arranged in chambers;

FIG. 3B is a simplified graphical representation of one embodiment of the encased probe array of FIGS. 1 and 2 including an inlet/outlet port fluidically connecting a first chamber to a second chamber and a fluid path to promote mixing;

FIG. 4A is a simplified graphical representation of one embodiment of the encased probe array of FIGS. 1 and 2 including construction thereof including a substrate and a secondary layer, wherein material is removed from both the substrate and secondary layer;

FIG. 4B is a simplified graphical representation of one embodiment of the encased probe array of FIGS. 1 and 2 including construction thereof including a substrate and secondary layer, wherein material is removed from the secondary layer;

FIG. 4C is a simplified graphical representation of one embodiment of the encased probe array of FIGS. 1 and 2 including construction thereof including a substrate and two secondary layers;

FIG. 5 is a functional block diagram of one embodiment of a first method of constructing the encased probe array of FIGS. 1 through 4; and

FIG. 6 is a functional block diagram of one embodiment of a second method of constructing the encased probe array of FIGS. 1 through 4.

### DETAILED DESCRIPTION

### a) GENERAL

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited. As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes. The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. patent application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. patent application Publication 20030096235), U.S. Ser. No. 09/910,292 (U.S. patent application Publication 20030082543), and U.S. Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Patents Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (U.S. Publication No. 20020183936), U.S. Ser. Nos. 10/065,856, 10/065,868, 10/328,818, 10/328,872, 10/423,403, and 60/482,389.

### b) DEFINITIONS

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer. Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers, initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being, attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM. Hybridization temperatures can be as low as 5.degree. C., but are typically greater than 22.degree. C., more typically greater than about 30.degree. C., and preferably in excess of about 37.degree. C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25 degrees-Celsius (° C.), e.g., 750 mM NaCl, 50 mM NaPhosphate, 5 mM EDTA, pH 7.4 (5× SSPE)and a temperature of from about 25 to about 30° C.

Hybridizations are usually performed under stringent conditions, for example, at a salt concentration of no more than 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

Hybridizing specifically to: refers to the binding, duplexing, or hybridizing of a molecule only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

### c) EMBODIMENTS OF THE PRESENT INVENTION

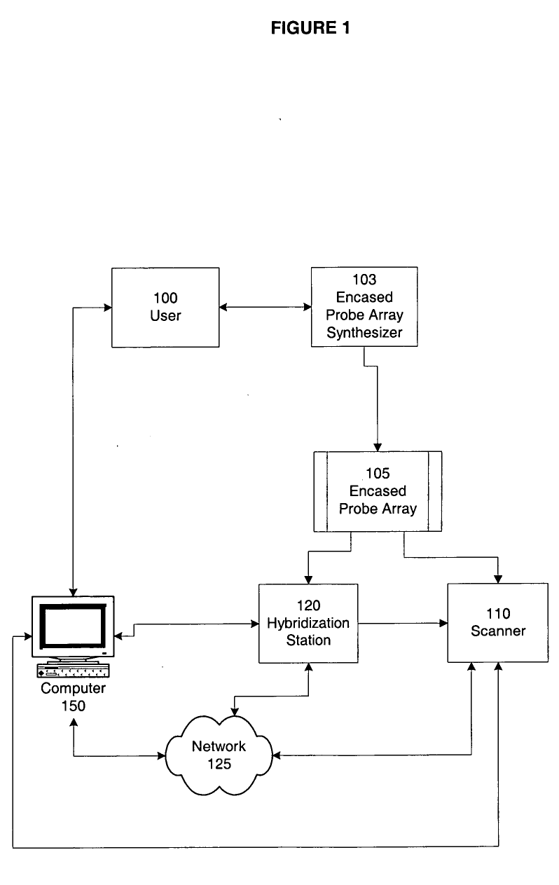

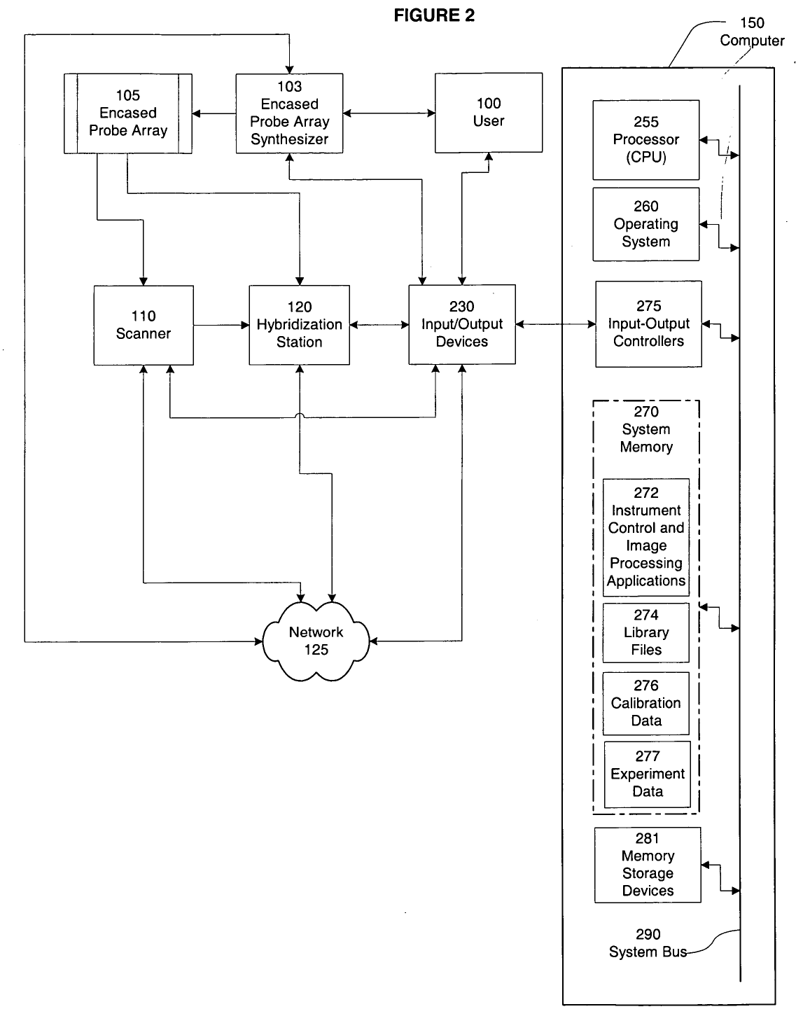

Computer 150: An illustrative example of computer 150 is provided in FIG. 1 and also in greater detail in FIG. 2. Computer 150 may be any type of computer platform such as a workstation, a personal computer, a server, or any other present or future computer. Computer 150 typically includes known components such as a processor 255, an operating system 260, system memory 270, memory storage devices 281, and input-output controllers 275, and input/output devices 230. Input/Output Devices 230 may include display devices that provides visual information, this information typically may be logically and/or physically organized as an array of pixels. A Graphical user interface (GUI) controller may also be included that may comprise any of a variety of known or future software programs for providing graphical input and output interfaces to a user, such as user 100, and for processing user inputs.

It will be understood by those of ordinary skill in the relevant art that there are many possible configurations of the components of computer 150 and that some components that may typically be included in computer 150 are not shown, such as cache memory, a data backup unit, and many other devices. Processor 255 may be a commercially available processor such as an Itanium® or Pentium® processor made by Intel Corporation, a SPARC® processor made by Sun Microsystems, an Athalon™ or Opteron™ processor made by AME corporation, or it may be one of other processors that are or will become available. Processor 255 executes operating system 260, which may be, for example, a Windows®-type operating system (such as Windows NT® 4.0 with SP6a, or Windows XP) from the Microsoft Corporation; a Unix® or Linux-type operating system available from many vendors or what is referred to as an open source; another or a future operating system; or some combination thereof. Operating system 260 interfaces with firmware and hardware in a well-known manner, and facilitates processor 255 in coordinating and executing the functions of various computer programs that may be written in a variety of programming languages. Operating system 260, typically in cooperation with processor 255, coordinates and executes functions of the other components of computer 150. Operating system 260 also provides scheduling, input-output control, file and data management, memory management, and communication control and related services, all in accordance with known techniques.

System memory 270 may be any of a variety of known or future memory storage devices. Examples include any commonly available random access memory (RAM), magnetic medium such as a resident hard disk or tape, an optical medium such as a read and write compact disc, or other memory storage device. Memory storage device 281 may be any of a variety of known or future devices, including a compact disk drive, a tape drive, a removable hard disk drive, or a diskette drive. Such types of memory storage device 281 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 270 and/or the program storage device used in conjunction with memory storage device 281.

In some embodiments, a computer program product is described comprising a computer usable medium having control logic (computer software program, including program code) stored therein. The control logic, when executed by processor 255, causes processor 255 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 275 could include any of a variety of known devices for accepting and processing information from a user, whether a human or a machine, whether local or remote. Such devices include, for example, modem cards, network interface cards, sound cards, or other types of controllers for any of a variety of known input devices. Output controllers of input-output controllers 275 could include controllers for any of a variety of known display devices for presenting information to a user, whether a human or a machine, whether local or remote. In the illustrated embodiment, the functional elements of computer 150 communicate with each other via system bus 290. Some of these communications may be accomplished in alternative embodiments using network or other types of remote communications.

As will be evident to those skilled in the relevant art, instrument control and image processing applications 272, if implemented in software, may be loaded into and executed from system memory 270 and/or memory storage device 281. All or portions of applications 272 may also reside in a read-only memory or similar device of memory storage device 281, such devices not requiring that applications/applications 272 first be loaded through input-output controllers 275. It will be understood by those skilled in the relevant art that applications 272, or portions of it, may be loaded by processor 255 in a known manner into system memory 270, or cache memory (not shown), or both, as advantageous for execution. Also illustrated in FIG. 2 are library files 274, calibration data 276, and experiment data 277 stored in system memory 270. For example, library files 274 could include data related to one or more probe arrays such as layout, content, or other related information. Also, calibration data 276 could include one or more values or other types of calibration data related to the calibration of scanner 110 or other instrument. Additionally, experiment data 277 could include data related to one or more experiments or assays such as the excitation ranges or values associated with one or more fluorescent labels.

Network 125 may include one or more of the many various types of networks well known to those of ordinary skill in the art. For example, network 125 may include what is commonly referred to as a TCP/IP network, or other type of network that may include the internet, or intranet architectures.

Instrument control and image processing applications 272: Instrument control and image processing applications 272 may be any of a variety of known or future image processing applications. Examples of applications 272 include Affymetrix® Microarray Suite, Affymetrix® GeneChip® Operating Software (hereafter referred to as GCOS), and Affymetrix® Jaguar™ software, noted above. Applications 272 may be loaded into system memory 270 and/or memory storage device 281 through one of input devices 230.

Embodiments of applications 272 include executable code being stored in system memory 270 of an implementation of computer 150. Applications 272 may provide a single interface for both the client workstation and one or more servers such as, for instance, GeneChip® Operating Software Server (GCOS Server). Applications 272 could additionally provide the single user interface for one or more other workstations and/or one or more instruments. In the presently described implementation, the single interface may communicate with and control one or more elements of the one or more servers, one or more workstations, and the one or more instruments. In the described implementation the client workstation could be located locally or remotely to the one or more servers and/or one or more other workstations, and/or one or more instruments. The single interface may, in the present implementation, include an interactive graphical user interface that allows a user to make selections based upon information presented in the GUI. For example, applications 272 may provide an interactive GUI that allows a user to select from a variety of options including data selection, experiment parameters, calibration values, probe array information. Applications 272 may also provide a graphical representation of raw or processed image data (described further below) where the processed image data may also include annotation information superimposed upon the image such as, for instance, base calls, features of the probe array, or other useful annotation information. Further examples of providing annotation information on image data are provided in U.S. Provisional Patent Application Serial No. 60/493,950, titled “System, Method, and Product for Displaying Annotation Information Associated with Microarray Image Data”, filed Aug. 8, 2003, which is hereby incorporated by reference herein in its entirety for all purposes.

In alternative implementations, applications 272 may be executed on a server, or on one or more other computer platforms connected directly or indirectly (e.g., via another network, including the Internet or an Intranet) to network 125.

Embodiments of applications 272 also include instrument control features. The instrument control features may include the control of one or more elements of one or more instruments that could, for instance, include elements of a fluidics station, what may be referred to as an autoloader, and scanner 110. The instrument control features may also be capable of receiving information from the one more instruments that could include experiment or instrument status, process steps, or other relevant information. The instrument control features could, for example, be under the control of or an element of the single interface. In the present example, a user may input desired control commands and/or receive the instrument control information via a GUI. Additional examples of instrument control via a GUI or other interface is provided in U.S. Provisional Patent Application Serial No. 60/483,812, titled “System, Method and Computer Software for Instrument Control, Data Acquisition and Analysis”, filed Jun. 30, 2003, which is hereby incorporated by reference herein in its entirety for all purposes.

In some embodiments, image data is operated upon by applications 272 to generate intermediate results. Examples of intermediate results include so-called cell intensity files (*.cel) and chip files (*.chp) generated by Affymetrix® GeneChip® Operating Software or Affymetrix® Microarray Suite (as described, for example, in U.S. patent application, Ser. Nos. 10/219,882, and 10/764,663, both of which are hereby incorporated herein by reference in their entireties for all purposes) and spot files (*.spt) generated by Affymetrix® Jaguar™ software (as described, for example, in PCT Application PCT/US 01/26390 and in U.S. patent applications, Ser. Nos. 09/681,819, 09/682,071, 09/682,074, and 09/682,076, all of which are hereby incorporated by reference herein in their entireties for all purposes). For convenience, the term “file” often is used herein to refer to data generated or used by applications 272 and executable counterparts of other applications, but any of a variety of alternative techniques known in the relevant art for storing, conveying, and/or manipulating data may be employed.

For example, applications 272 receives image data derived from a GeneChip® probe array and generates a cell intensity file. This file contains, for each probe scanned by scanner 110, a single value representative of the intensities of pixels measured by scanner 110 for that probe. Thus, this value is a measure of the abundance of tagged mRNA's present in the target that hybridized to the corresponding probe. Many such mRNA's may be present in each probe, as a probe on a GeneChip® probe array may include, for example, millions of oligonucleotides designed to detect the mRNA's. As noted, another file illustratively assumed to be generated by applications 272 is a chip file. In the present example, in which applications 272 include Affymetrix® GeneChip® Operating Software, the chip file is derived from analysis of the cell file combined in some cases with information derived from lab data and/or library files that specify details regarding the sequences and locations of probes and controls. The resulting data stored in the chip file includes degrees of hybridization, absolute and/or differential (over two or more experiments) expression, genotype comparisons, detection of polymorphisms and mutations, and other analytical results.

In another example, in which applications 272 includes Affymetrix® Jaguar™ software operating on image data from a spotted probe array, the resulting spot file includes the intensities of labeled targets that hybridized to probes in the array. Further details regarding cell files, chip files, and spot files are provided in U.S. patent application Ser. Nos. 09/682,098, 09/682,071, and 10/126,468, incorporated by reference above. As will be appreciated by those skilled in the relevant art, the preceding and following descriptions of files generated by applications 272 are exemplary only, and the data described, and other data, may be processed, combined, arranged, and/or presented in many other ways.

User 100 and/or automated data input devices or programs (not shown) may provide data related to the design or conduct of experiments. As one further non-limiting example related to the processing of an Affymetrix® GeneChip® probe array, the user may specify an Affymetrix catalogue or custom chip type (e.g., Human Genome U133 plus 2.0 chip) either by selecting from a predetermined list presented by GCOS or by scanning a bar code or RFID related to a probe array to read its type. GCOS may associate the probe array type with various scanning parameters stored in data tables including the area of the substrate that is to be scanned, the location of features on the substrate used for auto-focusing, the wavelength or intensity of laser light to be used in reading the hybridized probes associated with the probe array, and so on. As noted, applications 272 may apply some of this data in the generation of intermediate results. For example, information about the dyes may be incorporated into determinations of relative expression.

Those of ordinary skill in the related art will appreciate that one or more operations of applications 272 as described above may be performed by software or firmware associated with various instruments.

Scanner 110: Labeled targets hybridized to probe arrays may be detected using various devices, sometimes referred to as scanners, as described above with respect to methods and apparatus for signal detection. An illustrative device is shown in FIG. 1 as scanner 110. For example, scanners image the targets by detecting fluorescent or other emissions from labels associated with target molecules, or by detecting transmitted, reflected, or scattered radiation. A typical scheme employs optical and other elements to provide excitation light and to selectively collect the emissions.

For example, scanner 110 provides a signal representing the intensities (and possibly other characteristics, such as color) of the detected emissions or reflected wavelengths of light, as well as the locations on the substrate where the emissions or reflected wavelengths were detected. Typically, the signal includes intensity information corresponding to elemental sub-areas of the scanned substrate. The term “elemental” in this context means that the intensities, and/or other characteristics, of the emissions or reflected wavelengths from this area each are represented by a single value. When displayed as an image for viewing or processing, elemental picture elements, or pixels, often represent this information. Thus, in the present example, a pixel may have a single value representing the intensity of the elemental sub-area of the substrate from which the emissions or reflected wavelengths were scanned. The pixel may also have another value representing another characteristic, such as color, positive or negative image, or other type of image representation. The size of a pixel may vary in different embodiments and could include a 2.5 μm, 1.51 μm, 1.0 μm, or sub-micron pixel size. Two examples where the signal may be incorporated into data are data files in the form *.dat or *.tif as generated respectively by Affymetrix® Microarray Suite (described in U.S. patent application Ser. No. 10/219,882, incorporated above) or Affymetrix® GeneChip® Operating Software based on images scanned from GeneChip® arrays, and Affymetrix® Jaguar™ software (described in U.S. patent application Ser. No. 09/682,071, incorporated above) based on images scanned from spotted arrays. Examples of scanner systems that may be implemented with embodiments of the present invention include U.S. patent application Ser. No. 10/389,194, and U.S. Provisional Patent Application Serial No. 60/493,495 both of which are incorporated by reference above.

Hybridization station 120: FIG. 1 provides an illustrative example of hybridization station 120. Embodiments of station 120 may provide an instrument that implements one or more methods that could, for instance, include hybridizing one or more experimental samples to one or more probe arrays, and other processing steps such as labeling and washing.

Some embodiments of station 120 may include specialized instrumentation or alternatively may include general laboratory instruments that may be enabled to carry out one or more of the hybridizing and/or processing steps. Those of ordinary skill in the related art will appreciate that the description presented herein of station 120 is for the purposes of illustration only and is presented as an example, where one or more instruments and/or user intervention may be required to carry out the processes described below with respect to station 120. For example, embodiments of station 120 may be specialized specifically to interface with embodiments of encased probe array 105. Alternatively, it may be desirable in some implementations that encased probe array 105 be a low cost solution that can interface with standard laboratory instrumentation. In the present example, station 120 could include standard warming trays, baths, rockers, agitators, or similar types of instrumentation.

Some embodiments of station 120 could be enabled to introduce one or more samples, washes, buffers, stains, or other types of fluid into encased probe array 105 through one or more ports such as inlet/outlet port 315. For example, applications 272 may direct station 120 to add a specified volume of a particular sample to an associated implementation of encased probe array 105. Station 120 may remove the specified volume of sample from a reservoir and introduce the sample into encased probe array 105. The term “reservoir” as used herein could include a vial, tube, bottle, or some other container suitable for holding volumes of liquid. Also in the present example, station 120 may employ a vacuum/pressure source, valves, and means for fluid transport known to those of ordinary skill in the related art.

Similarly, some embodiments of station 120 could be enabled to remove used or waste fluids from encased probe array 105 by, for instance, creating a negative pressure or vacuum via interfacing a needle, pipette, or other type of transfer device with one or more of ports 315. Removal may, in some embodiments be aided by creating a positive pressure of gas or other fluids through a similar transfer device that may interface with one or more of ports 315 on the opposing side of encased probe array 105 and assist in “flushing” the fluid to be removed from encased probe array 105. Removed fluids may be stored in a waste reservoir or alternatively may be expelled from station 120 into another waste receptacle or drain.

Some embodiments of station 120 may provide an environment that promotes the hybridization of a biological target contained in a sample to the probes of the probe array. Some environmental conditions that affect the hybridization efficiency could include temperature, gas bubbles, agitation, oscillating fluid levels, or other conditions that could promote the hybridization of biological samples to probes. For example, station 120 may include a hybridization chamber that could, for instance, include a fluid bath for temperature control. In the present example, applications 272 may control the temperature of the fluid bath using methods known to those of ordinary skill in the related art and additionally may fluctuate the temperature according to parameters that may, for instance, be defined in experiment data 277.

Other environmental conditions that station 120 may provide may include a means to improve mixing of fluids within encased probe array 105. For example, ultrasonic agitation may provide vibration and fluidic movement that may improve the efficiency of hybridization of the sample to the probe array. Alternatively, station 120 may include a rocker or rotary mechanism that provides an oscillating surface, or holder that rotates about an axis that promotes movement and mixing of fluids within encased probe array 105 by inertial, centripetal, gravitational, or other related force.

Some embodiments of station 120 may also provide air or gas to encased probe array 105 for instance to promote the formation of a bubble or to promote the removal of fluid. For example, the gas bubble may include ambient air or other type of gas that may promote to mixing and redistribution of molecules within a fluid by moving with respect to the force of gravity for instance when encased probe array 105 is rotated about an axis.

Embodiments of station 120 may also perform what those of ordinary skill in the related art may refer to as post hybridization operations such as, for instance, washes with buffers or reagents, water, labels, or antibodies. For example, staining may include introducing molecules with fluorescent tags that selectively bind to the biological molecules or targets that have hybridized to the probes disposed in active area 305. In the present example, one or more fluorescently tagged molecules may bind to each probe/target pair where each additional fluorescent molecule that binds increases the intensity of emitted light during scanning. Also, the process of staining could include exposure of the hybridized probe array to molecules with fluorescent tags with different characteristics such as molecules that selectively bind to a specific hybridized probe target pairs, or a variety of fluorescent tags with different excitation and emission properties. Additional post-hybridization operations may, for example, include the introduction of what is referred to as a non-stringent buffer into encased probe array 105 to preserve the integrity of the hybridized array.

Station 120 may also perform operations that do not act directly upon a probe array. Such functions could include the management of fresh versus used reagents and buffers, experimental samples, or other materials utilized in hybridization or processing operations. Additionally, station 120 may include features for leak control and isolation from systems that may be sensitive to exposure to liquids. For example, a user may load a variety of experimental samples into station 120 that have unique experimental requirements. In the present example the samples may have barcode labels with unique identifiers associated with them. The barcode labels could be scanned with a hand held reader or alternatively station 120 could include an internal reader. Alternatively, other means of electronic identification could be used such as for instance what may be referred to as a radio frequency identifier (also referred to as RFID). The user may associate the identifier with the sample and store the data into one or more data files that for example could include experiment data 277 or library files 274. The sample may also be associated with a specific probe array type that is similarly stored.

Those of ordinary skill in the related art will also appreciate that similar identifiers and readers may be associated with various embodiments and implementations of encased probe array 105, where for example, applications 272 may be enabled to identify, track, and manage each of encased probe arrays 105.

Those of ordinary skill in the related art will appreciate that one or more of the operations described above may be performed by one or more instruments and/or by user 100. Examples of instruments for the processing and management of probe arrays are included in U.S. Pat. Nos. 6,114,122; and 6,391,623; 6,422,249 each of which is hereby incorporated by reference herein in its entirety for all purposes. Also, additional examples may also be found in U.S. patent application Ser. Nos. 10/712,860, and 10/684,160 both of which are also hereby incorporated by reference herein in their entireties for all purposes.

Encased Probe Array Synthesizer 103: As illustrated in FIGS. 1 and 2, embodiments of encased probe array 105 may be produced by encased probe array synthesizer 103. In the illustrative example of FIG. 1, encased probe array 105 is produced by encased probe array synthesizer 103 which receives instructions from computer 150. Specific examples of encased probe array 105 are provided in detail below.

Some embodiments of synthesizer 103 are enabled to produce a plurality of encased probe arrays 105 in a high throughput fashion. For example, multiple embodiments of encased probe arrays may be produced serially or in parallel where synthesizer 103 may produce each embodiment of encased probe array 105 according to one or more unique parameters that may, for instance, be defined in library files 274. In the present example, embodiments of encased probe arrays 105 may be produced by various methods of probe deposition onto a single substrate that then may be diced or divided by synthesizer 103 or by user 100 into individual implementations of encased probe array 105.

Additionally, embodiments of synthesizer 103 may be enabled to perform the methods of encasement described in further detail below. For example, encased probe array 105 may comprise at least two layers of glass, quartz, silica, or other material or various combinations thereof that may be fused, welded, glued, bonded, or otherwise attached by means known to those of skill in the related art. In the present example, synthesizer 103 may also perform the methods of defining one more chambers such as, for instance, by the removal of material from one or more of the layers or alternatively by casting or forming one or more of the layers with the chambers defined by the formation process.

Some embodiments of synthesizer 103 may be located remotely from user 100, scanner 110, and/or hybridization station 120 where, for example, each embodiment of encased probe arrays 105 may be transported to hybridization station 120 and scanner 110 for processing and analysis. Alternatively, synthesizer 103 may be directly associated with to hybridization station 120 and scanner 110 so that the process of producing, processing, and analyzing encased probe arrays 103 may be accomplished as a seamless process.

Some embodiments of synthesizer 103 may include systems and methods for reading and/or assigning barcode identifiers or other type of identifier such as, for instance, other means of electronic or optically based identification such as magnetic strips, what are referred to by those of ordinary skill in the related art as radio frequency identification (RFID), or other means of encoding information in a machine readable format. For example, synthesizer 103 may apply one or more barcodes each associated with a barcode identifier to each implementation of encased probe array 105 using techniques known to those of ordinary skill in the related art such as, for instance, affixing a label, printing, or other type of method for labeling. In the present example, the barcode identifier may be comprised of one or more elements that could include unique identifiers, probe array type, lot number, expiration date, user identifiers, one or more experimental parameters, or other type of associated information.

In some embodiments, computer 150 may assign one or more elements of a barcode identifier for each implementation of encased probe array 105 such as, for instance, a unique identifier, and create database records, experiment files, or other type of data structure that may contain the barcode identifiers, one or more elements of the barcode identifiers, and associated information that may be retrieved based, at least in part, upon one or more of the elements of the barcode identifiers. Computer 150 may then forward the database records and/or experiments files to a LIMS system or other remote server or storage device.

Additional examples of probe array spotting and synthesis systems are s provided in Patent Cooperation Treaty Application Ser. No. PCT/US02/13883, filed May 2, 2002, which is hereby incorporated by reference herein in its entirety for all purposes; and in U.S. patent application Ser. No. 10/684,160, incorporated by reference above.

Those of ordinary skill in the related art will appreciate that the instruments and functions described with respect to encased probe array synthesizer 150 are for the purpose of illustration only and should not be considered limiting in any way. For example, the described functions need not be performed by a single instrument but rather may be performed by a plurality of instruments performing various steps that may occur at various points in time. Similarly in the case of a plurality of instruments, the instruments need not be located in close proximity to one another but rather one or more may be located remotely from a first instrument.

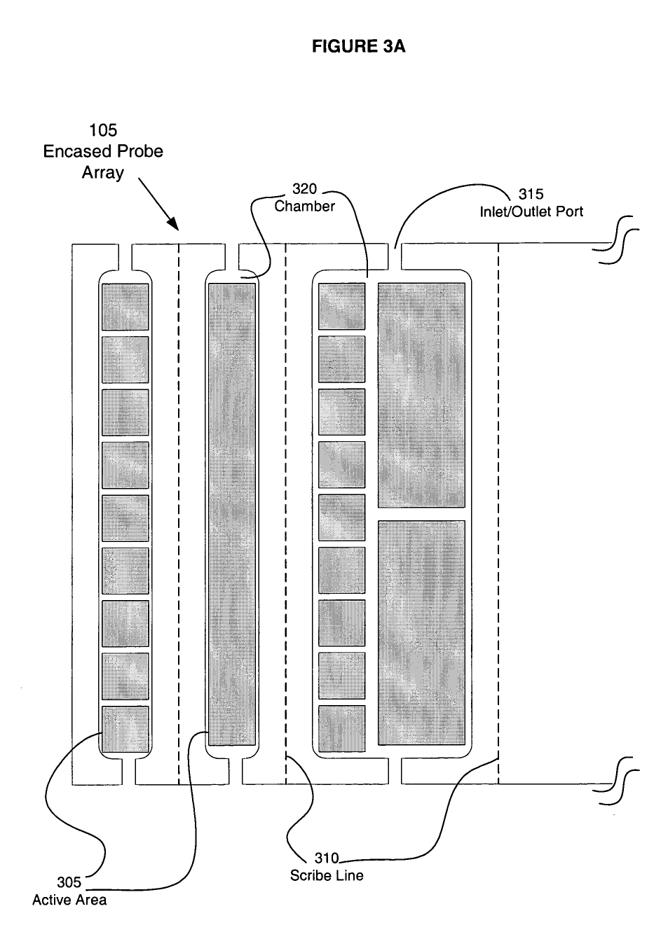

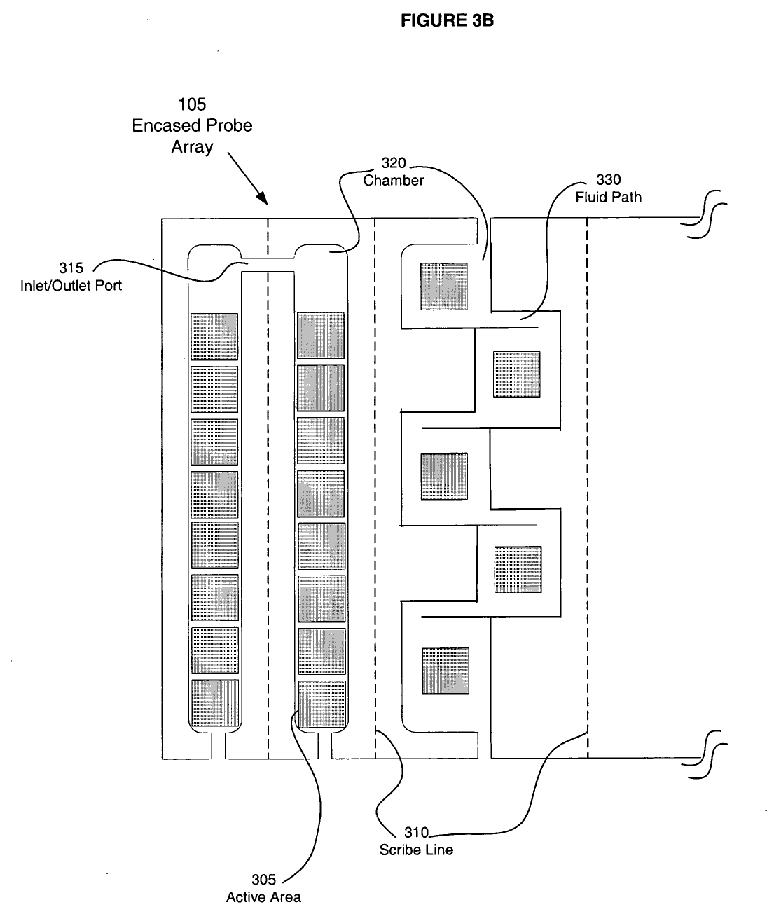

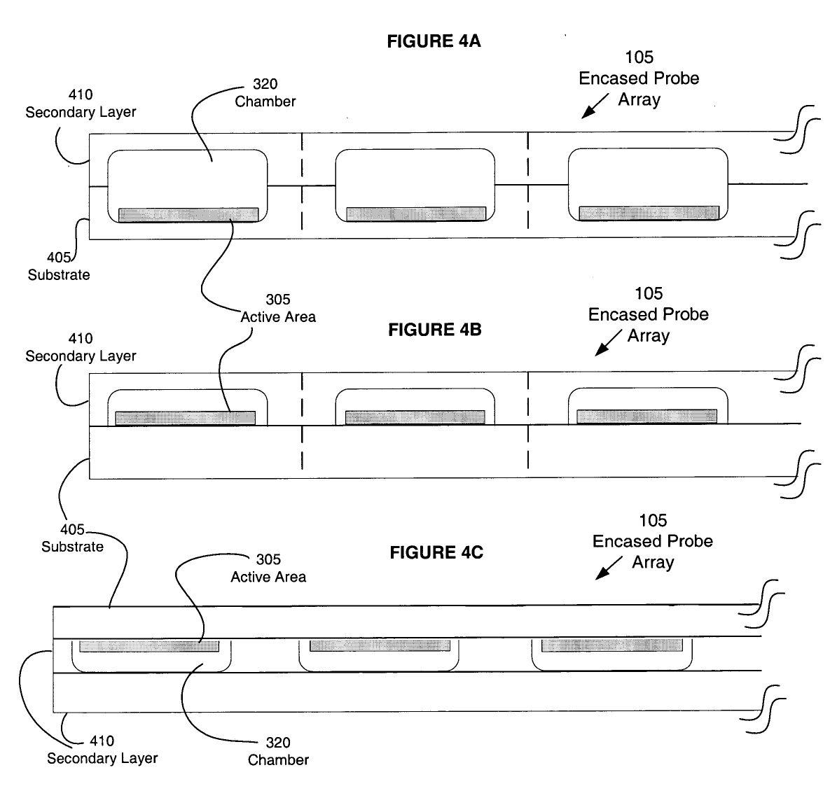

Encased Probe Array 105: Various embodiments of encased probe array 105 are graphically illustrated in FIGS. 3A through 4C. More specifically FIGS. 3A and 3B present a perspective of encased probe array 105 that is substantially normal to the plane defined by the substantially planar substrate of encased probe array 105. FIGS. 4A through 4C present a “cutaway view” perspective of encased probe array 105 that is substantially parallel to the plane defined by the substantially planar substrate of encased probe array 105. The embodiments of encased probe array 105 present a low cost, flexible, and scaleable alternative to housed probe arrays currently available. In the present example, encased probe array 105 may be produced inexpensively, enabled to interface with generally available laboratory instrumentation, and be flexible with respect to probe content, number and size of active areas, as well as the size of each embodiment of encased probe array 105. Also, encased probe array 105 provides a sealed environment that protects the probes disposed in one or more active areas from insult. For example, encased probe array 105 could include the area defined by a standard probe array synthesis wafer such as, for instance, a wafer that is 5 inches long and 5 inches wide. Encased probe array 105 may also include smaller implementations where a large embodiment is reduced to several smaller independent implementations such as by dicing or other means of separation.

Some embodiments of encased probe array 105 may include one or more enclosed chambers that may be defined, at least in part, by a substantially planar “bottom layer” that could for instance include substrate 405 upon which one or more probes are disposed, and one or more secondary layers 410 that form one or more substantially planar “top layers”. Also, various methods may be employed to define the one or more chambers in one or more of the bottom or top layers. Examples of such methods, as well as methods of forming embodiments of encased probe array 105 are presented in greater detail below.

FIGS. 3A and 3B provide illustrative examples of encased probe array 105 that include one or more active areas 305, and one or more chambers 320. Each of active areas 305 include a plurality of probes disposed upon a substrate in accordance with systems and methods described above with respect to polymer array synthesis and Nucleic acid arrays. For example, each embodiment of encased probe array 105 may include one or more of chambers 320 that in turn may also include one or more active areas 305. Both active areas 305 and chamber 320 may be variable in size and shape. Also in the present example, each chamber 320 may include at least one inlet/outlet port 315.

FIG. 3A presents an illustrative example of encased probe array 105 that includes a plurality of chambers 320, where each of chambers 320 may contain one or more active areas 305. In the example of FIG. 3A each of chambers 320 includes a number of active areas that may vary in number and size. Those of ordinary skill will appreciate that the number, size and probe content of active area 305 may include any of the many possible combinations known in the art. FIG. 3A also includes scribe line 310 that may indicate a preferred region that includes an area between implementations of chamber 320 that may, for instance, provide an indication or means for dicing or separating encased probe array 105 into smaller implementations. For example, scribe line 310 may be etched into one or more of layers 405 and/or 410 of encased probe array 105 where line 310 includes a “scribe line” that enables user 100 to easily break along line 310 by hand as is known in the art of glass cutting. In the present example, scribe line 310 may include a reference mark used by encased probe array synthesizer 103 or other similar instrument for separation by other mechanical means such as dicing. The reference mark may be included on one or more of layers 405 or 410.

FIG. 3A also provides an example where each embodiment of chamber 320 includes two inlet/outlet ports 315 that each provides an opening for an interface with transfer devices. Also illustrated in FIG. 3B, inlet/outlet ports 315 may be employed to fluidically connect embodiments of chamber 320. For example, inlet/outlet port 315 may provide a fluidic connection between two chambers where port 315 crosses scribe line 310. It may be desirable in some circumstances to fluidically couple multiple implementations of chamber 320 for particular processing steps where each chamber may later be separated along scribe line 310, where each separate implementation may subsequently be treated differently. In the present example, once individual embodiments are separated along scribe line 310 each are then fluidically independent of each other.

Those of ordinary skill in the related art will appreciate that each of ports 315 are sealable, where the act of sealing may prevent the loss of liquid or gas. For example, ports 315 may be sealed by methods known in the art such as, for instance, with wax, rubber or plastic stoppers, or other means that provides an outward force against the walls of ports 315. Alternatively, some embodiments of ports 315 may include mechanical means such as threads, notches, or keyed elements that accept particular sealing members with corresponding threads, keys or notches where the act of sealing could also in some embodiments be enhanced by O-rings or gaskets.

Some embodiments of encased probe arrays may also include features such as fluid path 330, that may promote turbulent flow of liquids or gasses along a path from one embodiment of port 315 used as an inlet port to a second embodiment of port 315 used as an outlet port. In addition to fluid path 330, encased probe array 105 may include one or more additional elements to promote mixing and the redistribution of molecules in a fluid or gas such as pillars, posts, textured surfaces, or other similar elements known in the related art.

Embodiments of encased probe array 105 may be constructed by various means and include various materials. It may be desirable in many implementations that substrate layer 405, and one or more secondary layers 410 be optically transparent. It may also be desirable in the same or additional implementations that layers 405 and 410 exhibit other qualities as well such as, for instance, lacking or having a low level of fluorescent properties and light scattering properties. For example, it may be desirable in some implementations that substrate 405 and one or more secondary layers 410 are transparent to enable scanner 110 to provide an excitation light either through substrate 405 for what me be referred to as “backside scanning”, through one or more secondary layers 410 for what may be referred to as “frontside scanning”, or both. In the present example, scanner 110 may include a detector to collect emitted light from the same side that provides the excitation light or alternatively scanner 110 may include a detector on the opposite side of where one or more filters may be employed to block excitation wavelengths of light directed at the detector.

Alternatively, in some embodiments it may be desirable that one or more of layers 405 or 410 be opaque or reflective where the opaque layer blocks, absorbs, or reflects substantially all wavelengths of light. In yet another possible embodiment, one or more layers could be substantially reflective at a first range of wavelengths and substantially transmissive at one or more second range of wavelengths. For example, one or more coatings could be applied to one or more of layers 405 or 410, where the one or more coatings could provide one or more desired light absorbing, scattering, reflecting, or transmitting properties. Additional examples of coatings associated with probe arrays are included in U.S. Provisional Patent Application Serial No. 60/565,442, titled “System and Method for Increased Light Collection”, filed Apr. 26, 2004, which is hereby incorporated by reference herein in its entirety for all purposes.

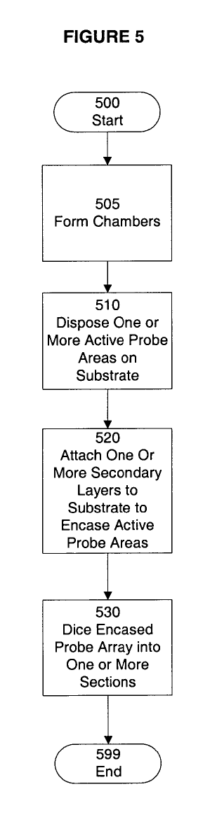

FIGS. 4A through 4C and FIGS. 5 and 6 provide illustrative examples of the possible construction and related methods of constructing encased probe arrays 105. FIG. 4A provides an example of a method of forming one or more chambers according to step 505 where material is removed from both substrate 405 and a secondary layer 410 by methods such as etching that could include dry (anisotropic), wet (isotropic), or mechanical etching, or other means known in the art. In the present example, the removal of material may be used to form each embodiment of chamber 320, inlet/outlet port 315, and/or fluid path 330. Also, the probes associated with each embodiment of active area 305 may be deposited, or synthesized on substrate 405 in an area associated with an embodiment of chamber 320 as illustrated in step 510 of FIG. 5. Further, step 520 illustrates the step of attaching substrate 405 and secondary layer 410 to one another by for instance, employing what may be referred to as anodic bonding techniques, adhesives, welds, or other means known in the related art, thereby encasing each active area 305 within a chamber 320.

In an alternative example, the act of disposing one or more active probe areas according to step 510 may be performed directly upon substrate 405 without the removal of material as illustrated in FIG. 4B. In the example of FIG. 4B the a method of forming one or more chambers according to step 505 includes removing material from secondary layer 410 by means as described above with respect to FIG. 4A to create chamber 320, inlet/outlet port 315, and/or fluid path 330. Also in the present example, the method of attaching substrate 405 and secondary layer 410 is performed as described above with respect to step 520.

In yet another example, active area 305 may be disposed upon substrate 405 per step 510, and the method of forming one or more chambers according to step 505 includes a first implementation of secondary layer 410, where chamber 320, inlet/outlet port 315, and/or fluid path 330 is molded, pressed, or formed by other means known to those of skill in the related art. The first implementation of secondary layer 410 and a second implementation of secondary layer 410 may be attached to substrate 405 according to step 520, where the first molded implementation of secondary layer 410 forms an intermediate layer between substrate 405 and the second implementation of secondary layer 410.

In the examples described above, encased probe array 105 may be separated according to step 530 and as previously described, into smaller implementations of encased probe array 105. Also in each of the above examples, substrate 405 could be constructed of quartz silica, glass, plastic such as what may be referred to as Polymethylmethacrylate (sometimes referred to as PMMA), or other rigid substrate. Similarly, secondary layer 410 may also be constructed of quartz silica, glass, plastic, or other rigid and non-rigid materials.

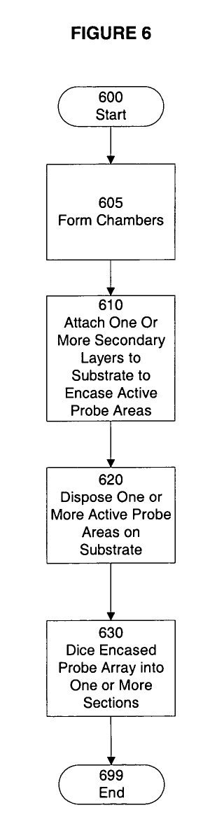

Other methods of constructing encased probe array 105 may also be employed such as the method as illustrated in FIG. 6. Those of ordinary skill in the art will appreciate that the method of FIG. 6 may be applied to any of the construction embodiments previously described and illustrated in FIGS. 4A through 4C. One or more chambers may be formed according to step 605 as described above with respect to step 505. Additionally, each of substrate 405 and one or more secondary layers 410 are attached according to step 610 prior to the synthesis or deposition of active area 305, where the method of attachment may be the same as those described with respect to step 520. The act of attaching forms chamber 320, inlet/outlet port 315, and/or fluid path 330 that may be used as a flow cell for probe synthesis chemistry. For example, step 620 may include a method of light directed probe synthesis as described above where light is directed through either substrate 405 to one or more secondary layers 410 for what may be referred to as a de-protection step and one or more chemicals such as nucleic acids may be flowed through the flow cell for building probes. Thus in the present example, active area 305 is disposed upon substrate 405 after the step of encasement.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

Also, the functions of several elements may, in alternative embodiments, be carried out by fewer, or a single, element. Similarly, in some embodiments, any functional element may perform fewer, or different, operations than those described with respect to the illustrated embodiment. Also, functional elements shown as distinct for purposes of illustration may be incorporated within other functional elements in a particular implementation. Also, the sequencing of functions or portions of functions generally may be altered. Certain functional elements, files, data structures, and so on may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. An encased probe array, comprising:
a first layer including one more active areas disposed thereon, wherein each of the one or more active areas includes a plurality of probes each enabled to hybridize a biological molecule;
one or more second layers attached to the first layer; and
one or more chambers formed from at least one of the one or more second layers, wherein each of the one or more active areas is associated with at least one of the one or more chambers.

2. The encased probe array of claim 1, wherein:
the first layer comprises a quartz layer.

3. The encased probe array of claim 1, wherein:
the one or more second layers comprises a quartz layer.

4. The encased probe array of claim 1, wherein:
the one or more second layers comprises a PMMA layer.

5. The encased probe array of claim 1, wherein:
each of the first layer and one or more second layers is optically transparent.

6. The encased probe array of claim 1, wherein:
the one or more chambers are formed by removal of material from at least one of the one or more second layers.

7. The encased probe array of claim 1, further comprising:
the one or more chambers are formed from the first layer.

8. The encased probe array of claim 7, wherein:
the one or more chambers are formed by removal of material from the first layer and at least one of the one or more second layers.

9. The encased probe array of claim 1, wherein:
the one or more chambers are defined by molding at least one of the one or more second layers.

10. The encased probe array of claim 1, further comprising:
one or more ports fluidically coupled to at least one of the one or more chambers.

11. The encased probe array of claim 10, wherein:
at least one of the one or more ports provides an interface with a transfer device.

12. The encased probe array of claim 10, wherein:
at least one of the one or more ports provides a fluidic connection between a first chamber and a second chamber.

13. The encased probe array of claim 10, wherein:
the one or more ports are sealable.

14. The encased probe array of claim 1, further comprising:
one or more scribe lines each positioned between a first chamber and a second chamber of the one or more chambers.

15. The encased probe array of claim 14, wherein:
at least one of the one or more scribe lines is etched into a layer from the group consisting of the first layer, and the one or more second layers.

16. The encased probe array of claim 14, wherein:
at least one of the one or more scribe lines include a reference mark on a layer from the group consisting of the first layer, and the one or more second layers.

17. The encased probe array of claim 14, wherein:
each of the one or more scribe lines is used for dicing.

18. The encased probe array of claim 1, further comprising:
one or more fluid paths each providing a fluidic connection between a first chamber and a second chamber of the one or more chambers.

19. The encased probe array of claim 18, wherein:
each of the one or more fluid paths further provides a means for mixing a fluid.

20. The encased probe array of claim 1, wherein:
the first layer and the one or more second layers is substantially planar.

21. A method of producing an encased probe array, comprising:
disposing one more active areas on a first layer, wherein each of the one or more active areas includes a plurality of probes each enabled to hybridize a biological molecule;
forming one or more chambers from at least one of one or more second layers, wherein each of the one or more active areas is associated with at least one of the one or more chambers; and
attaching one or more second layers to the first layer.

22. The method of claim 21, wherein:
the step of forming comprises removing material from at least one of the one or more second layers.

23. The method of claim 21, wherein:
the step of forming comprises molding at least one of the one or more second layers.

24. The method of claim 21, further comprising:
forming one or more chambers from the first layer.

25. The method of claim 24, wherein:
the step of forming comprises removing material from the first layer.

26. The method of claim 24, wherein:
the step of forming comprises molding the first layer.

27. The method of claim 21, wherein:
the step of disposing comprises synthesizing each of the plurality of probes on the first layer.

28. The method of claim 21, further comprising:
fluidically coupling a first chamber and a second chamber of the one or more chambers.

29. The method of claim 21, further comprising:
positioning a scribe line between a first chamber and a second chamber of the one or more chambers.

30. The method of claim 29, further comprising:
dicing the attached first and second layers based, at least in part, upon the scribe line.

31. A system for scanning encased probe arrays, comprising:
one or more encased probe arrays, wherein each encased probe array comprises:
a first layer including one more active areas disposed thereon, wherein each of the one or more active areas includes a plurality of probes each enabled to hybridize a biological molecule;
one or more second layers attached to the first layer; and
one or more chambers formed from at least one of the one or more second layers, wherein each of the one or more active areas is associated with at least one of the one or more chambers;
a scanner to acquire an image of each of the active areas; and
a computer comprising an image analysis application stored and executed thereon to analyze the image.
